(19) United States
(12) Patent Application Publication
(10) Pub. No.: US 2008/0065009 A1
(43) Pub. Date: Mar. 13, 2008

(54) Drug Delivery in the Coronary Sinus

(75) Inventor: Shmuel Ben-Muvhar, Emek Beit Shean (IL)

Correspondence Address: DARBY & DARBY P.C., P.O. BOX 770, Church Street Station, New York, NY, 10008-0770, US

(73) Assignee: Neo-Vasc Medical Ltd., Or Yehuda (IL)

(21) Appl. No.: 11/573,626

(22) PCT Filed: Jul. 31, 2005
(86) PCT No.: PCT/IL05/00810

§ 371 Date: Oct. 11, 2007

Related U.S. Application Data
(60) Provisional application No. 60/601,068, filed on Aug. 12, 2004.

(51) Int. Cl. A61M 25/04; A61M 25/10; A61M 29/00

(52) U.S. Cl. 604/101.01; 604/104; 604/174; 604/175; 604/96.01

(57) ABSTRACT

A method for delivering a therapeutic substance in a coronary sinus (28) of a patient includes inserting a catheter (30) through a vein (22) into a right atrium (26) of the patient, and advancing the catheter from the right atrium into the coronary sinus. A resilient anchor (34), which is fixed to the catheter, is released so as to anchor the catheter in the coronary sinus without blocking blood flow through the coronary sinus. The therapeutic substance is then administered from the catheter into the coronary sinus.

### CROSS-REFERENCE TO RELATED APPLICATION

This application claims the benefit of U.S. Provisional Patent Application 60/601,068, filed Aug. 12, 2004, which is incorporated herein by reference.

### BACKGROUND OF THE INVENTION

Methods and devices for delivery of drugs in the coronary sinus are known in the art. Such methods and devices are disclosed, for example in the following U.S. patents: U.S. Pat. No. 6,416,493, U.S. Pat. No. 4,927,412, U.S. Pat. No. 5,487,730 and U.S. Pat. No. 6,709,415, whose disclosures are incorporated herein by reference. All the devices described in these patents are designed for short-term use, such as for retrograde perfusion of cardioplegia solution during cardiac surgery.

### SUMMARY OF THE INVENTION

Embodiments of the present invention provide catheters and anchoring mechanisms for such catheters that can be used to administer therapeutic substances into the coronary sinus over extended periods, typically over the course of many hours or even days. The anchoring mechanism holds the catheter securely in place within the coronary sinus, generally without substantially blocking the venous flow through the coronary sinus into the right atrium and without significant trauma to the coronary sinus or other anatomical structures. In the embodiments described hereinbelow, the catheter is anchored securely notwithstanding the flow of blood and the widening shape of the coronary sinus leading into the right atrium. The catheter may then be used to administer therapeutic substances into the coronary sinus. This arrangement is particularly useful, for example, in cell therapy, as well as in other cardiac drug treatments.

There is therefore provided, in accordance with an embodiment of the present invention, a method for delivering a therapeutic substance in a coronary sinus of a patient, including:

inserting a catheter through a vein into a right atrium of the patient, and advancing the catheter from the right atrium into the coronary sinus;

releasing a resilient anchor, which is fixed to the catheter, so as to anchor the catheter in the coronary sinus without blocking blood flow through the coronary sinus; and

administering the therapeutic substance from the catheter into the coronary sinus.

There is also provided, in accordance with an embodiment of the present invention, a method for delivering a therapeutic substance in a coronary sinus of a patient, including:

providing a catheter including a distal end that includes multiple parallel leaves formed from a pliable material;

enclosing the catheter within a flexible sheath;

while the catheter is enclosed within the flexible sheath, inserting the catheter and the sheath through a vein into a right atrium of the patient, and advancing the catheter and the sheath from the right atrium into the coronary sinus;

shifting the catheter distally relative to the sheath so as to release the parallel leaves from the sheath and to cause the leaves to bend outward so that the leaves anchor the catheter in the coronary sinus; and

administering the therapeutic substance from the catheter into the coronary sinus.

There is additionally provided, in accordance with an embodiment of the present invention, a method for delivering a therapeutic substance in a coronary sinus of a patient, including:

inserting a catheter through a vein into a right atrium of the patient, and advancing the catheter from the right atrium into the coronary sinus;

inflating one or more balloons, which are fixed to the catheter, so as to anchor the catheter in the coronary sinus without blocking blood flow through the coronary sinus; and

administering the therapeutic substance from the catheter into the coronary sinus.

There is further provided, in accordance with an embodiment of the present invention, a method for delivering a therapeutic substance in a coronary sinus of a patient, including:

implanting a stent within the coronary sinus;

inserting a catheter through a vein into a right atrium of the patient, and advancing the catheter from the right atrium into the coronary sinus;

releasing an anchor, which is fixed to the catheter, so as to anchor the catheter to the stent; and

administering the therapeutic substance from the catheter into the coronary sinus.

There is moreover provided, in accordance with an embodiment of the present invention, apparatus for delivering a therapeutic substance in a coronary sinus of a patient, including:

a catheter, which is adapted to be inserted through a blood vessel of the patient into the coronary sinus, and which has a lumen for administration of the therapeutic substance therethrough into the coronary sinus; and

a resilient anchor, which is fixed to the catheter, so as to anchor the catheter in the coronary sinus without blocking blood flow through the coronary sinus.

In some embodiments, the catheter has a distal end and a channel, and the resilient anchor includes at least one resilient wire, which is arranged to be passed through the channel so as to protrude from the distal end of the catheter and engage a blood vessel wall. In one embodiment, the catheter has at least first and second channels, and the at least one resilient wire includes at least first and second wires, which are arranged to be passed respectively through the first and second channels so as to engage the blood vessel wall at two or more locations on different sides of the catheter.

In some embodiments, the at least one resilient wire includes a shape memory material, which is held straight within the channel and is pre-formed to bend into a predetermined shape upon protruding from the distal end so as to engage the blood vessel wall. Typically, the predetermined shape includes at least one of a loop, a spiral and a zigzag shape. Additionally or alternatively, the predetermined shape includes a bend, and wherein the at least one resilient wire is adapted to bend from the catheter into a side vessel that joins the coronary sinus and to engage the wall of the side vessel.

In a disclosed embodiment, the channel has multiple exit holes at different locations along the distal end of the catheter, and is arranged to permit an operator of the apparatus to choose one of the exit holes, and to advance the at least one resilient wire through the chosen exit hole so as to engage the blood vessel wall at a desired location.

In some embodiments, the channel has a proximal entrance, and the at least one resilient wire has a proximal end and includes a handle, which is fixed to the proximal end of the at least one resilient wire for use by an operator of the apparatus in manipulating the wire in the channel, so that the handle engages the proximal entrance of the channel when the at least one resilient wire is fully extended from the distal end of the catheter. Additionally or alternatively, the catheter includes a locking mechanism for locking the at least one resilient wire in place at the proximal end of the channel after the at least one resilient wire has engaged the blood vessel wall so as to prevent relative axial movement between the catheter and the at least one resilient wire. The locking mechanism may include a spring clip, which is arranged to press against the at least one resilient wire, or a locking screw, which is arranged to engage the at least one resilient wire within the channel.

In other embodiments, the catheter includes a flexible sheath, which is arranged to enclose the catheter while containing the resilient anchor, and to be advanced with the catheter through the blood vessel, wherein the flexible sheath is adapted to permit the catheter to be shifted distally relative to the sheath so as to release the anchor, causing the anchor to engage a blood vessel wall. In some of these embodiments, the anchor includes one or more resilient wires, which are pre-formed so as to spring outward into a predetermined shape when released from the sheath. Typically, the predetermined shape includes at least one of a spiral, a loop and a hook shape.

Additionally or alternatively, the catheter includes a distal end, which includes multiple parallel leaves formed from a resilient material, and which serve as the resilient anchor, such that upon shifting the catheter distally relative to the sheath, the leaves bend outward and engage the wall of the coronary sinus. Typically, the leaves are arranged, upon bending outward, to at least partially block blood flow through the coronary sinus. In a disclosed embodiment, the leaves are constructed so as to deform in response to excessive blood pressure in the coronary sinus, thereby permitting blood to flow out of the coronary sinus.

In another embodiment, the resilient anchor is arranged to anchor the catheter by engaging a stent implanted in the coronary sinus.

There is furthermore provided, in accordance with an embodiment of the present invention, apparatus for delivering a therapeutic substance in a coronary sinus of a patient, including:

a catheter, which is adapted to be inserted through a blood vessel of the patient into the coronary sinus, and which has a lumen for administration of the therapeutic substance therethrough into the coronary sinus, and which includes a distal end including multiple parallel leaves formed from a pliable material; and

a flexible sheath, which is arranged to enclose the catheter so as to contain the flexible leaves, and to be advanced with the catheter through a blood vessel into the coronary sinus, wherein the flexible sheath is adapted to permit the catheter to be shifted distally relative to the sheath so as to release the parallel leaves from the sheath and to cause the leaves to bend outward so that the leaves anchor the catheter in the coronary sinus.

In one embodiment, the leaves are adapted to bend outward under a pressure of blood flow in the coronary sinus. In another embodiment, the apparatus includes a balloon at the distal end of the catheter, which is inflatable so as to push the leaves outward. Typically, the leaves are arranged, upon bending outward, to at least partially block the blood flow through the coronary sinus.

There is also provided, in accordance with an embodiment of the present invention, apparatus for delivering a therapeutic substance in a coronary sinus of a patient, including:

a catheter, which is adapted to be inserted through a blood vessel of the patient into the coronary sinus, and which has a lumen for administration of the therapeutic substance therethrough into the coronary sinus; and

one or more balloons, which are fixed to the catheter, and which are inflatable so as to anchor the catheter in the coronary sinus without blocking blood flow through the coronary sinus.

The apparatus may include at least one additional balloon, which is fixed to the catheter and is inflatable so as to adjustably block the blood flow through the coronary sinus while the catheter is anchored in the coronary sinus.

There is additionally provided, in accordance with an embodiment of the present invention, apparatus for delivering a therapeutic substance in a coronary sinus of a patient, including:

a stent, which is adapted to be implanted within the coronary sinus;

a catheter, which is adapted to be inserted through a blood vessel of the patient into the coronary sinus, and which has a lumen for administration of the therapeutic substance therethrough into the coronary sinus; and

an anchor, which is fixed to the catheter, and is arranged to anchor the catheter to the stent.

In one embodiment, the stent contains a constricted opening, and the catheter includes a distal end that is adapted to pass through the constricted opening in the stent, and the anchor includes a balloon, which is attached to the distal end of the catheter and is arranged to be inflated after the distal end of the catheter has passed through the constricted opening. Alternatively or additionally, the anchor includes one or more wires, which are arranged to be advanced from the distal end of the catheter so as to engage the stent, and the catheter is arranged to be advanced distally along the wires to a position within the stent.

The present invention will be more fully understood from the following detailed description of the embodiments thereof, taken together with the drawings in which:

### BRIEF DESCRIPTION OF THE DRAWINGS

FIG. 1 is a schematic, cutaway view of a heart into which a catheter is inserted, in accordance with an embodiment of the present invention;

FIG. 2 is a schematic detail view of the distal end of a catheter within the coronary sinus, in accordance with an embodiment of the present invention;

FIGS. 3-6 are schematic detail views of the distal ends of catheters, in accordance with alternative embodiments of the present invention;

FIG. 7 is a schematic, pictorial illustration showing an arrangement for manipulating a wire at the proximal end of a catheter, in accordance with an embodiment of the present invention;

FIGS. 8A and 8B are schematic, pictorial illustrations showing an arrangement for fixing a wire at the proximal end of a catheter in accordance with another embodiment of the present invention;

FIG. 9 is a schematic, pictorial illustration showing an arrangement for securing the position of a wire inside a channel in a catheter, in accordance with a further embodiment of the present invention;

FIGS. 10A and 10B are sectional illustrations of the catheter shown in FIG. 9, taken along the line marked X-X in FIG. 9;

FIGS. 11A and 11B are schematic, side views of a catheter within the coronary sinus, illustrating a method for anchoring the distal end of the catheter, in accordance with another embodiment of the present invention;

FIG. 12A is a schematic side view of a catheter in the coronary sinus, illustrating another method for anchoring the distal end of the catheter, in accordance with an alternative embodiment of the present invention;

FIG. 12B is a schematic, sectional view of the catheter shown in FIG. 12A, taken along the line marked XIIB-XIIB;

FIGS. 12C and 12D are schematic, sectional views of the distal end of a catheter, in accordance with alternative embodiments of the present invention;

FIGS. 13A and 13B are schematic side views of the distal end of a catheter, in accordance with another embodiment of the present invention; and

FIG. 14 is a schematic side view of a catheter in the coronary sinus, illustrating yet another method for anchoring the distal end of the catheter, in accordance with an embodiment of the present invention.

### DETAILED DESCRIPTION OF EMBODIMENTS

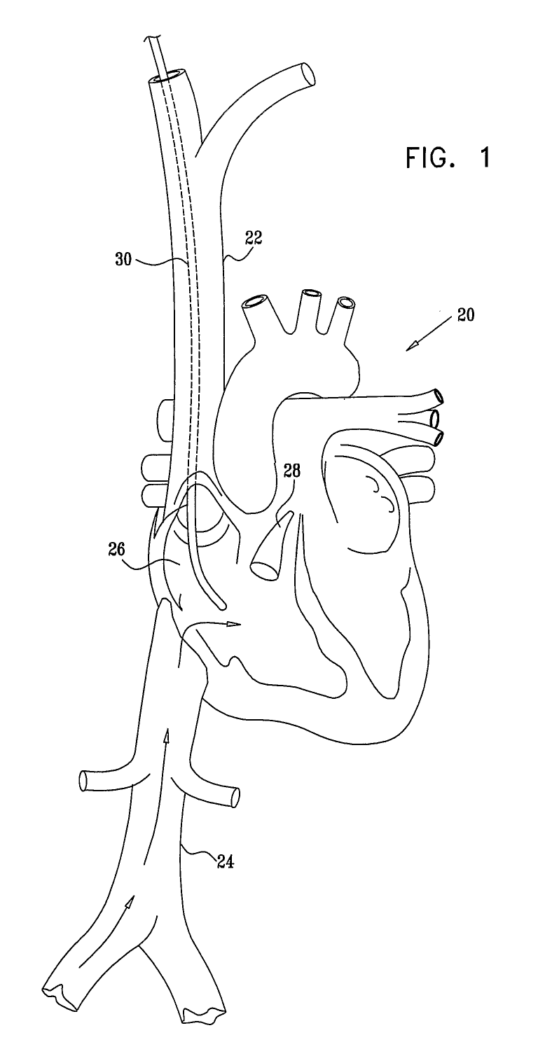

FIG. 1 is a schematic, cutaway view of a heart 20, having a coronary sinus 28 into which a catheter 30 is inserted, in accordance with an embodiment of the present invention. The catheter in this embodiment is inserted percutaneously, typically via the jugular vein, into the superior vena cava 22 and thence into right atrium 26. Alternatively, the catheter may be inserted into the right atrium by other routes, as are known in the art, such as through the femoral vein and the inferior vena cava 24. The distal end of the catheter is maneuvered into coronary sinus 28, and is then anchored in place using methods and anchoring mechanisms such as those described hereinbelow.

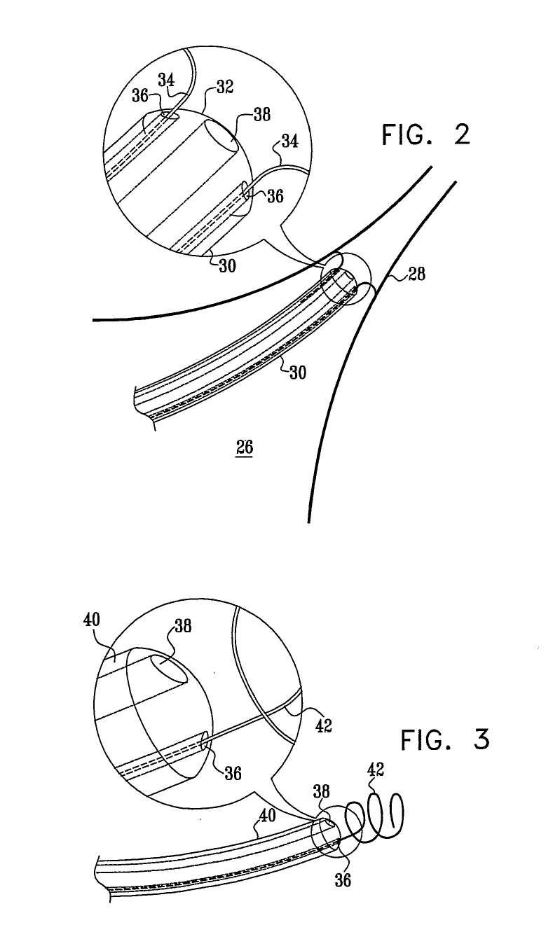

FIG. 2 is a schematic detail view of a distal end 32 of catheter 30 within coronary sinus 28, in accordance with an embodiment of the present invention. Two anchoring wires 34 protrude from respective channels 36. Although in the embodiments pictured in this patent application, channels 36 are contained within the body of the catheter, these channels may alternatively be joined externally to the catheter tube. Furthermore, although each of the figures in this patent application shows a certain number of anchoring elements (such as the two wires 34 in FIG. 2), it will be understood that the principles of these anchoring mechanisms may be implemented using larger or smaller numbers of anchoring elements in each case.

Anchoring wires 34 comprise a resilient biocompatible material, typically a shape memory material, such as Nitinol. During insertion of the catheter through the venous system and into coronary sinus 28, wires 34 are contained inside channels 36 and do not protrude outside distal end 32. The wires extend all the way through catheter 30 and are accessible at the proximal end of the catheter. Once the distal end of catheter 30 has been suitably positioned in the coronary sinus, wires 34 are pushed forward from the proximal end, and are thus caused to advance out of the distal end of channels 36. The wires are pre-molded so that when freed from the confines of the narrow channels, the wires bend outward, as shown in FIG. 2, and engage the wall of the coronary sinus. The force of engagement is sufficient to anchor catheter 30 in place so as to withstand the flow of blood from the coronary veins via sinus 28 into atrium 26, without causing injury or significant trauma to the wall of the coronary sinus. Despite partial obstruction of sinus 28 by catheter 30, the blood from the coronary veins is still able to flow past the catheter into atrium 26.

While catheter 30 is thus anchored in place, a therapeutic substance, such as a drug or cell therapy medium, is injected into the coronary sinus through a lumen 38 in catheter 30. Alternatively, the therapeutic substances may be pre-loaded into a suitable reservoir within distal end 32 of catheter 30, and then released from the reservoir when the catheter is anchored in place. Typically, the therapeutic substance is released in a gradual, continuous dosage, or alternatively, in a sequence of individual doses that are spaced over time. Catheter 30 need not be removed from the heart during the treatment period. Upon completion of the treatment, wires 34 are retracted in the proximal direction through channels 36, and catheter 30 is removed from the body.

FIG. 3 is a schematic detail view of the distal end of another catheter 40, in accordance with an alternative embodiment of the present invention. In this case, a resilient wire 42 is pre-molded so as to form a loop, zigzag or spiral shape when advanced distally out of channel 36. The pre-formed shape is slightly larger in transverse dimension than the diameter of coronary sinus 28 at the target location of the catheter. As a result, wire 42 engages the wall of coronary sinus 28 and thus anchors the catheter in place.

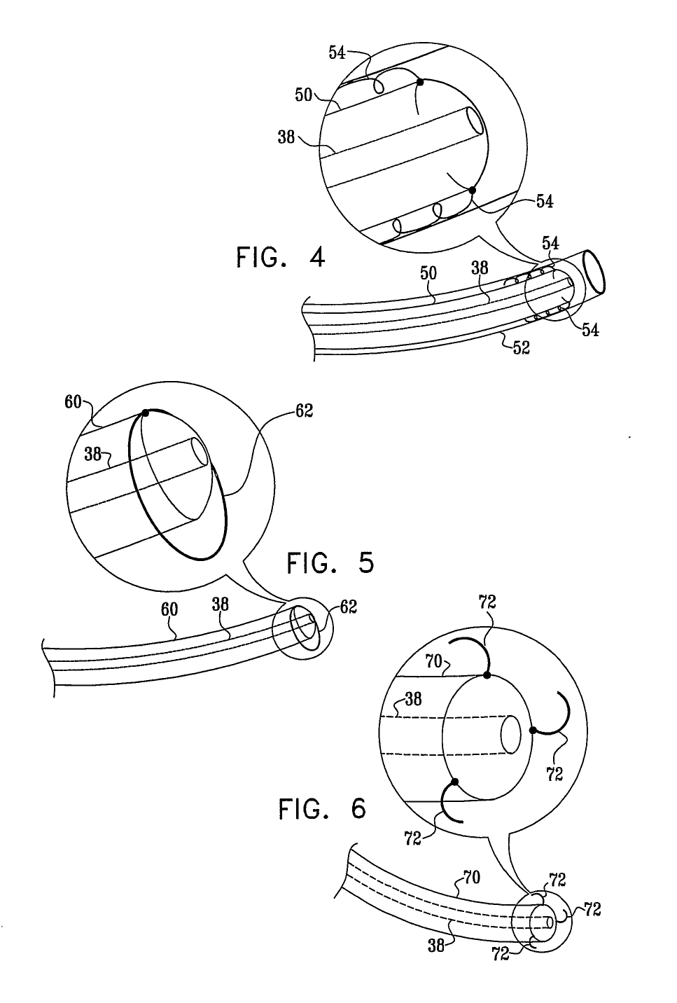

FIG. 4 is a schematic detail view of the distal end of a catheter 50, in accordance with yet another embodiment of the present invention. Anchors 54 are fixed externally to the distal end of catheter 50. As in the preceding embodiments, the anchors comprise resilient wires. During insertion through the vasculature and into the heart, catheter 50 is contained inside a flexible sheath 52. Anchors 54 are thus contained and held inside the sheath, against the sides of catheter 50. Once the distal end of sheath 52 has reached the coronary sinus, catheter 50 is shifted relative to the sheath in the distal direction (by advancing the catheter and/or withdrawing the sheath), so that the distal end of the catheter protrudes out of the distal end of the sheath into the coronary sinus. Anchors 54 are pre-molded so that when released from sheath 52, the wires spring outward and engage the wall of the coronary sinus. Sheath 52 may then be withdrawn entirely from the body, or it may alternatively be left in place during the treatment.

In order to remove catheter 50 from the coronary sinus, sheath 52 is advanced distally over the distal end of the catheter, thus gathering anchors 54 back into the sheath and releasing the catheter. The sheath, with the catheter and anchors held inside, is then withdrawn from the body.

FIGS. 5 and 6 are schematic detail views of the distal ends of catheters 60 and 70, in accordance with further embodiments of the present invention. Catheters 60 and 70 are designed to be inserted into the coronary sinus and withdrawn therefrom inside a sheath, such as sheath 52, as in the embodiment of FIG. 4. The embodiments of FIGS. 5 and 6 illustrate different types of anchors: Catheter 60 has a loop anchor 62, which is compressed inside the sheath and then expands to its full circular shape when released. Catheter 70 has hook anchors 72, which similar spring outward when released from the sheath.

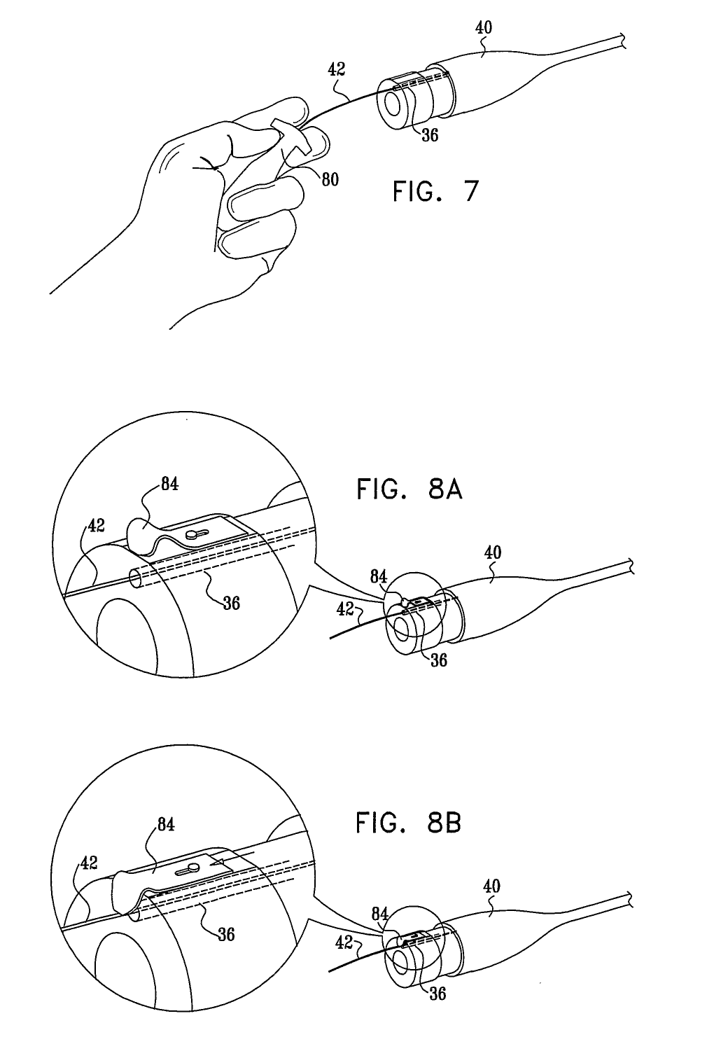

FIG. 7 is a schematic, pictorial illustration showing the proximal end of catheter 40 (whose distal end was shown in FIG. 3), in accordance with an embodiment of the present invention. The proximal end of wire 42 is fixed to a handle 80. While the catheter is maneuvered into place through the vascular system and into the coronary sinus, handle 80 is distanced from the proximal end of the catheter, so that wire 42 does not protrude from the distal end of the catheter. Once the catheter is properly positioned in the coronary sinus, handle 80 is advanced so that it engages the proximal end of the catheter. In this position, the distal end of wire 42 extends out of the distal end of the catheter, as shown in FIG. 3. Handle 80 may lock against the proximal entrance of channel 36 in this forward position in order to prevent relative movement between the catheter and the wire during treatment.

FIGS. 8A and 8B are schematic, pictorial illustrations showing the proximal end of catheter 40 in accordance with another embodiment of the present invention. In this case, a spring clip 84 is provided in order to prevent relative axial movement between catheter 40 and wire 42 during treatment. When clip 84 is retracted, as shown in FIG. 8A, wire 42 is free to move in channel 36. After wire 42 has been advanced distally and has engaged the walls of the coronary sinus, clip 84 slides in the proximal direction and exerts pressure on wire 42, as shown in FIG. 8B, in order to hold the wire in place relative to the catheter.

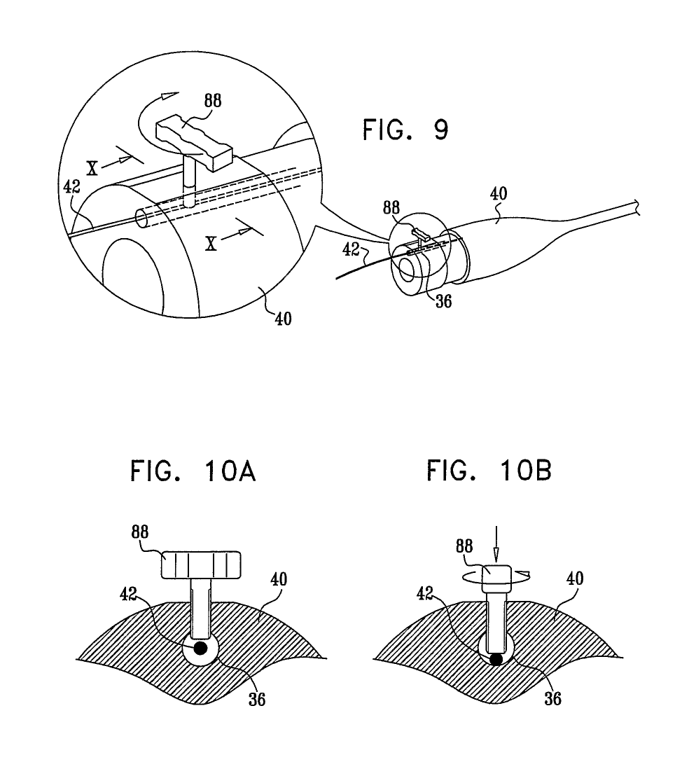

FIGS. 9, 10A and 10B schematically illustrate another arrangement for securing the position of wire 42 inside channel 36, in accordance with a further embodiment of the present invention. FIG. 9 is a pictorial illustration, while FIGS. 10A and 10B are sectional illustrations taken along the line X-X in FIG. 9. A thumbscrew 88 rotates in a threaded hole at the proximal end of catheter 40. As shown in FIGS. 10A and 10B, turning the screw alternately releases wire 42 to move within channel 36 or secures the wire in place.

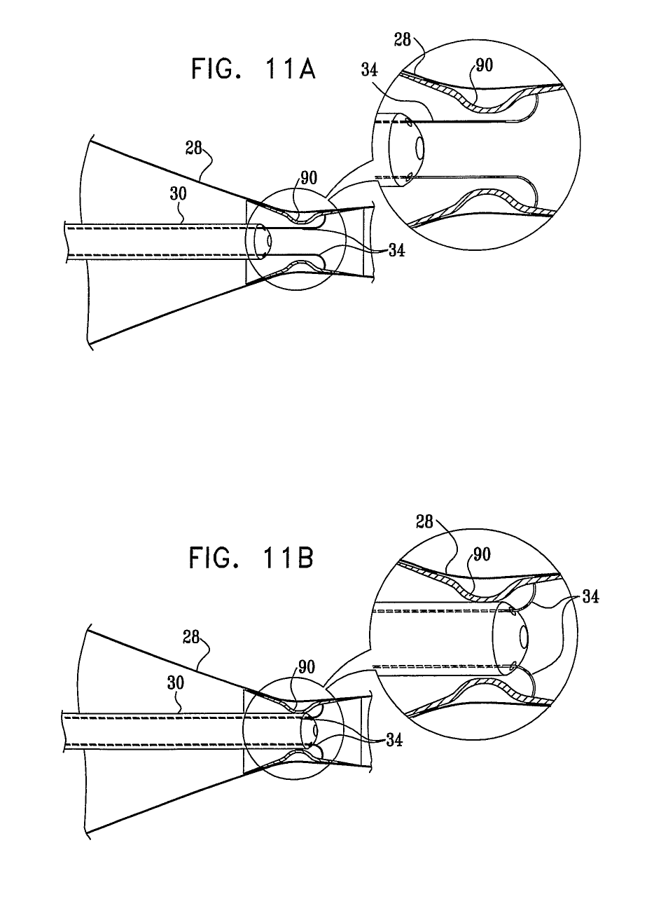

FIGS. 11A and 11B are schematic, side views showing another method for anchoring the distal end of catheter 30 (FIG. 2) within coronary sinus 28, in accordance with an embodiment of the present invention. This method relies on the presence of a stent 90 within the coronary sinus. Stents of this sort are described, for example, in U.S. Patent Application Publication US2003/0097172, whose disclosure is incorporated herein by reference. As described in this patent, such stents may be implanted in the coronary sinus in order to narrow the diameter of the coronary sinus and thus restrict blood flow from the coronary veins into the heart. This sort of restriction has been shown to promote angiogenesis. In the present embodiment, however, stent 90 is used simply as a platform for anchoring catheter 30, and the therapeutic properties of the stent are not material to the present invention. Thus, the principles of the present embodiment could be implemented using other types of vascular stents, as well.

As shown in FIGS. 11A and 11B, once catheter 30 is positioned in coronary sinus 28, wires 34 are extended distally out of the catheter and engage stent 90. Typically, the stent itself comprises wires or struts, against which wires 34 catch and hold the catheter in place. During drug delivery, catheter 30 may be advanced distally along the wires, as shown in FIG. 11B, so that the distal end of the catheter is positioned inside the narrowest part of stent 90. In this position, blood flow from the coronary veins into the heart is temporarily blocked, thus facilitating retrograde perfusion of therapeutic substances injected into the coronary sinus via lumen 38 of catheter 30.

In between treatments, catheter 30 may be withdrawn in the proximal direction to the position shown in FIG. 11A. In this position, blood can flow out of the coronary sinus into the right atrium, but the catheter can be easily returned to the position shown in FIG. 11B for subsequent treatments.

FIGS. 12A and 12B schematically illustrate another method for anchoring a catheter 92 within stent 90 in coronary sinus 28, in accordance with an alternative embodiment of the present invention. FIG. 12A is a side view of the catheter, while FIG. 12B is a sectional view taken along the line XIIB-XIIB in FIG. 12A. In this embodiment, a donut-shaped balloon 94 is fixed to the distal end of catheter 92. During insertion and maneuvering of the catheter, balloon 94 remains deflated. After the distal end of catheter 92 has been inserted through the constricted opening of stent 90, as shown in FIG. 12A, balloon 94 is inflated via an inflation tube 95 to hold the catheter in place, as well as blocking the flow of blood through the coronary sinus.

FIGS. 12C and 12D are schematic, sectional views through the distal end of catheter 92, in accordance with alternative embodiments of the present invention. In these embodiments, a number of separate balloons 96 are attached peripherally to the distal end of the catheter. As in the preceding embodiment, the balloons remain deflated during insertion, maneuvering, and withdrawal of the catheter, and are inflated through respective inflation tubes 95 in order to anchor the catheter when the catheter is in place. (Alternatively, a single inflation tube may be used for multiple balloons.) Balloons 96 may be used in conjunction with stent 90, as in the preceding embodiment, or they may alternatively be used to anchor the catheter without assistance of a stent. Although FIGS. 12C and 12D show two and four balloons, respectively, the principles of these embodiments may alternatively be applied using one or three balloons or using a greater number of balloons.

The embodiment of FIG. 12C is advantageous in that balloons 96 can anchor catheter 92 in place, without trauma to the blood vessel wall, while still permitting blood to flow past the catheter, into the right atrium. Alternatively, in the embodiment of FIG. 12D, the balloons may be inflated sufficiently to substantially block the blood flow. In this configuration, the inflation pressure of the balloons may be controlled in order to regulate the amount of blood flow, thus increasing or decreasing the venous back-pressure. For example, the two balloons above and below the catheter in FIG. 12D may be inflated to relatively high pressure in order to anchor the catheter in place, and the pressure in the balloons to the sides of the catheter may be adjusted up and down until the desired flow rate or back-pressure is achieved.

FIGS. 13A and 13B are schematic side views of the distal end of a catheter 100, in accordance with still another embodiment of the present invention. The anchors for the catheter in this case are produced by making the distal end of catheter 100 from a material, comprising multiple parallel leaves 102. (Although three leaves are shown in the figure, the catheter may alternatively comprise two leaves or four or more leaves of this sort, or even a single leaf protruding to one side.)

In one embodiment, leaves 102 are pre-formed from a resilient material so as to bend outward when released, as shown in FIG. 13B. During insertion through the vasculature and into the heart, catheter 100 is contained inside flexible sheath 52, as shown in FIG. 13A. When catheter 100 is in place within coronary sinus 28, the sheath is withdrawn, causing leaves 102 to bend outward and thus anchor the catheter within the coronary sinus, as shown in FIG. 13B. In this configuration, the leaves block at least a portion of the blood flow from the coronary sinus into the right atrium. The degree of blockage may be controlled by appropriate selection of the sizes of catheter 100 and leaves 102 and of the precise location in coronary sinus 28 at which the leaves are released. Complete blockage of the coronary sinus is often undesirable, since it may compromise blood flow to the heart muscle and cause infarction. To avoid this sort of danger, leaves 102 may be made sufficiently flexible so that overpressure in the coronary sinus will cause the leaves to deform, thereby permitting blood to flow out between the leaves and relieving the blockage.

In an alternative embodiment, leaves 102 are formed from a pliable material. As a result, when catheter 100 is in place in the coronary sinus, and sheath 52 is withdrawn, the pressure of the flowing blood in the coronary sinus pushes the leaves outward into the configuration shown in FIG. 13B. The leaves then anchor the catheter in place and partially block the blood flow, as noted above. Alternatively, a balloon (not shown in the figures) at the center of the distal end of catheter 100 may be inflated in order to push the leaves apart. Further alternatively or additionally, catheter 100 may be anchored in place by other means, such as one of the anchor wires described above, and leaves 102 may be opened for the purpose of providing a desired degree of blockage of the blood flood.

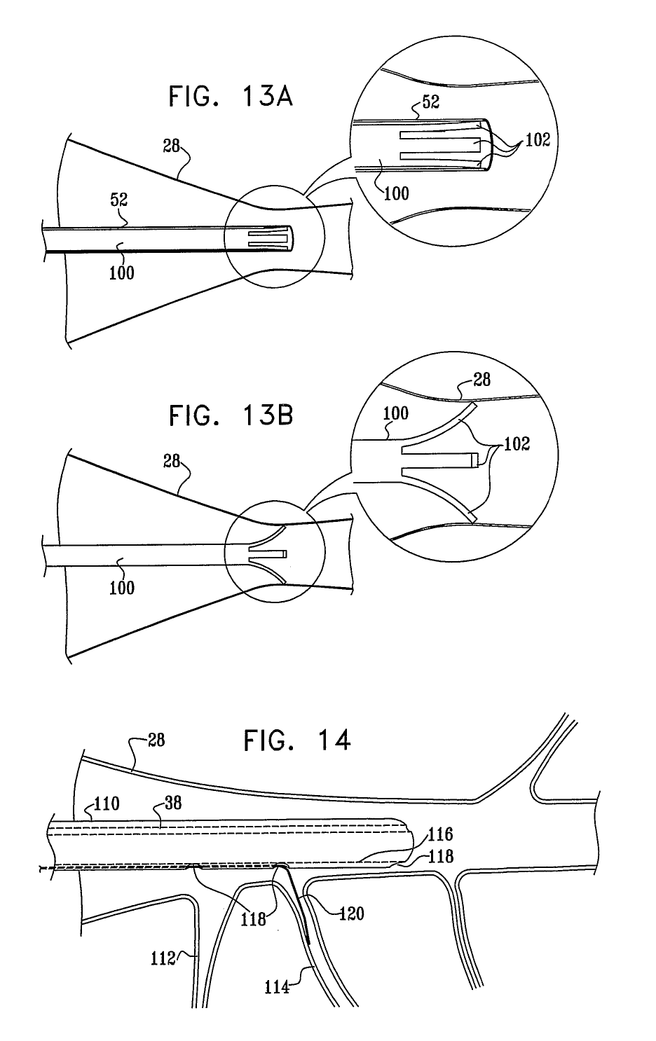

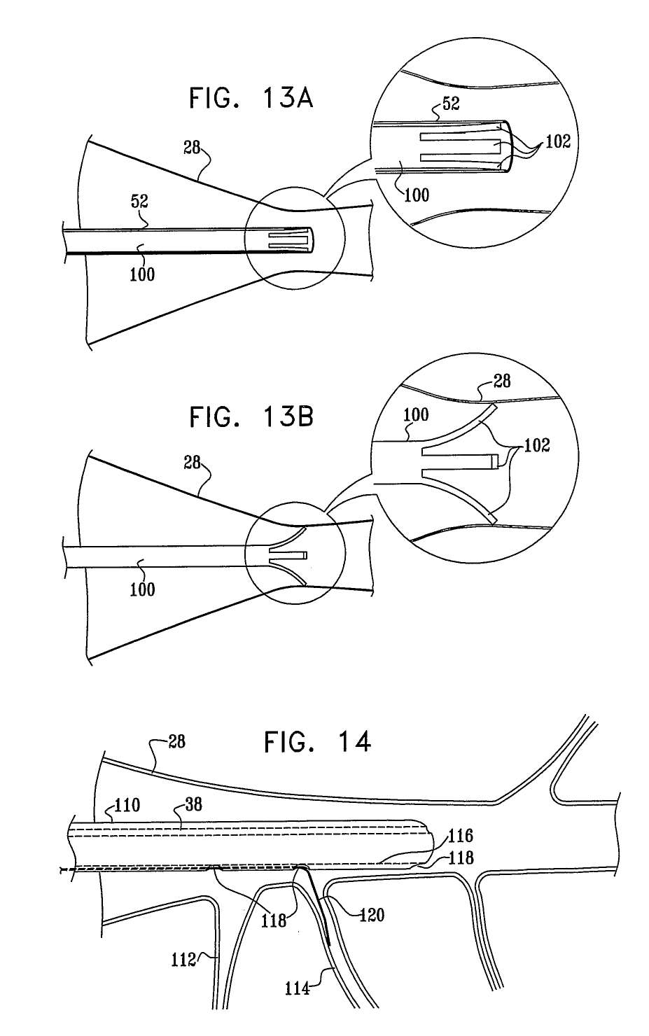

FIG. 14 is a schematic side view of a catheter 110 in coronary sinus 28, illustrating yet another method for anchoring the distal end of the catheter, in accordance with an embodiment of the present invention. Catheter 110 comprises a channel 116 with multiple openings 118 in the vicinity of the distal end of the catheter. A resilient wire 120, which is pre-formed in a bent shape, is contained inside channel 116 during insertion of catheter 110 through the vasculature. Once the distal end of the catheter has been suitably positioned in the coronary sinus, wire 120 is pushed forward from the proximal end, and is thus caused to advance out of one of holes 118. Catheter 110 may comprise a suitable control mechanism (not shown) for individually opening the different holes so that wire 120 exits channel 116 through the desired hole. Alternatively, the operator of the catheter may determine the hole through which the wire is to exit by first aligning the end of the wire with the desired hole, and then rotating the wire within the channel until the pre-bent tip of the wire slides out through the hole.

The advantage of providing multiple holes 118 in channel 116 is that it gives the operator more flexibility in deciding where and how to anchor catheter 110. For example, the operator may align one of holes 118 with a side vessel of the coronary sinus, such as small cardiac vein 112 or middle cardiac vein 114 (typically under fluoroscopic imaging), and may then push wire 120 out through the hole so that it enters the chosen side vessel. The bent tip of the wire engages the side vessel, as shown in FIG. 14, and thus anchors the catheter in place. In this manner, the operator can determine the depth of penetration of the catheter within the coronary sinus. (Even if channel 116 has only a single exit hole, the operator can still exert some control over the depth of penetration by appropriate choice of the side vessel into which wire 120 is to be inserted.)

Although the embodiments described hereinabove relate specifically to anchoring of catheters in the coronary sinus, the principles of the present invention may similarly be applied in anchoring catheters in other locations in the cardiovascular system, as well as anchoring of invasive probes of other types in other body passages and cavities. It will thus be appreciated that the embodiments described above are cited by way of example, and that the present invention is not limited to what has been particularly shown and described hereinabove. Rather, the scope of the present invention includes both combinations and subcombinations of the various features described hereinabove, as well as variations and modifications thereof which would occur to persons skilled in the art upon reading the foregoing description and which are not disclosed in the prior art.

1. A method for delivering a therapeutic substance in a coronary sinus of a patient, comprising:
inserting a catheter through a vein into a right atrium of the patient, and advancing the catheter from the right atrium into the coronary sinus;
releasing a resilient anchor, which is fixed to the catheter, so as to anchor the catheter in the coronary sinus without blocking blood flow through the coronary sinus; and
administering the therapeutic substance from the catheter into the coronary sinus.

2. The method according to claim 1, wherein releasing the resilient anchor comprises advancing at least one resilient wire through a channel associated with the catheter so that the at least one resilient wire protrudes from a distal end of the catheter and engages a blood vessel wall.

3. The method according to claim 2, wherein advancing the at least one resilient wire comprises advancing at least first and second wires through respective first and second channels so as to engage the blood vessel wall at two or more locations on different sides of the catheter.

4. The method according to claim 2, wherein the at least one resilient wire comprises a shape memory material, which is held straight within the channel and is pre-formed to bend into a predetermined shape upon protruding from the distal end so as to engage the blood vessel wall.

5. The method according to claim 4, wherein the predetermined shape comprises at least one of a loop, a spiral and a zigzag shape.

6. The method according to claim 4, wherein the predetermined shape comprises a bend, and wherein advancing the at least one resilient wire comprises aligning the at least one resilient wire to enter a side vessel that joins the coronary sinus and to engage the wall of the side vessel.

7. The method according to claim 2, wherein the channel has multiple exit holes at different locations along the distal end of the catheter, and wherein advancing the at least one resilient wire comprises choosing one of the exit holes, and advancing the at least one resilient wire through the chosen exit hole so that the at least one resilient wire engages the blood vessel wall at a desired location.

8. The method according to claim 2, wherein advancing the at least one resilient wire comprises advancing a handle, which is fixed to a proximal end of the at least one resilient wire, so that the handle engages a proximal entrance of the channel.

9. The method according to claim 2, and comprising locking the at least one resilient wire in place at a proximal end of the channel after the at least one resilient wire has engaged the blood vessel wall so as to prevent relative axial movement between the catheter and the at least one resilient wire.

10. The method according to claim 9, wherein locking the at least one resilient wire comprises actuating a spring clip to press against the at least one resilient wire.

11. The method according to claim 9, wherein locking the at least one resilient wire comprises turning a locking screw to engage the at least one resilient wire within the channel.

12. The method according to claim 2, wherein the catheter comprises a distal end, which comprises multiple parallel leaves, and comprising opening the leaves outward to at least partially block the blood flow through the coronary sinus during administration of the therapeutic substance.

13. The method according to claim 12, wherein opening the leaves comprises inflating a balloon at the distal end of the catheter in order to push the leaves apart.

14. The method according to claim 1, wherein inserting the catheter comprises enclosing the catheter within a flexible sheath while advancing the catheter through the vein, wherein the flexible sheath contains the resilient anchor, and wherein releasing the resilient anchor comprises shifting the catheter distally relative to the sheath so as to release the anchor, causing the anchor to engage a blood vessel wall.

15. The method according to claim 14, wherein the anchor comprises one or more resilient wires, which are preformed so as to spring outward into a predetermined shape when released from the sheath.

16. The method according to claim 15, wherein the predetermined shape comprises at least one of a spiral, a loop and a hook shape.

17. The method according to claim 14, wherein the catheter comprises a distal end, which comprises multiple parallel leaves formed from a resilient material, and which serve as the resilient anchor, such that upon shifting the catheter distally relative to the sheath, the leaves bend outward and engage the wall of the coronary sinus.

18. The method according to claim 17, wherein the leaves are arranged, upon bending outward, to at least partially block blood flow through the coronary sinus.

19. The method according to claim 18, wherein the leaves are constructed so as to deform in response to excessive blood pressure in the coronary sinus, thereby permitting blood to flow out of the coronary sinus.

20. The method according to claim 1, wherein releasing the resilient anchor comprises anchoring the catheter to a stent implanted in the coronary sinus.

21. A method for delivering a therapeutic substance in a coronary sinus of a patient, comprising:
providing a catheter comprising a distal end that comprises multiple parallel leaves formed from a pliable material;
enclosing the catheter within a flexible sheath;
while the catheter is enclosed within the flexible sheath, inserting the catheter and the sheath through a vein into a right atrium of the patient, and advancing the catheter and the sheath from the right atrium into the coronary sinus;
shifting the catheter distally relative to the sheath so as to release the parallel leaves from the sheath and to cause the leaves to bend outward so that the leaves anchor the catheter in the coronary sinus; and
administering the therapeutic substance from the catheter into the coronary sinus.

22. The method according to claim 21, wherein the leaves are adapted to bend outward under a pressure of blood flow in the coronary sinus.

23. The method according to claim 21, and comprising inflating a balloon at the distal end of the catheter so as to push the leaves outward.

24. The method according to claim 21, wherein the leaves are arranged, upon bending outward, to at least partially block the blood flow through the coronary sinus.

25. A method for delivering a therapeutic substance in a coronary sinus of a patient, comprising:
inserting a catheter through a vein into a right atrium of the patient, and advancing the catheter from the right atrium into the coronary sinus;
inflating one or more balloons, which are fixed to the catheter, so as to anchor the catheter in the coronary sinus without blocking blood flow through the coronary sinus; and
administering the therapeutic substance from the catheter into the coronary sinus.

26. The method according to claim 25, and comprising inflating at least one additional balloon, which is fixed to the catheter, so as to adjustably block the blood flow through the coronary sinus while the catheter is anchored in the coronary sinus.

27. A method for delivering a therapeutic substance in a coronary sinus of a patient, comprising:
implanting a stent within the coronary sinus;
inserting a catheter through a vein into a right atrium of the patient, and advancing the catheter from the right atrium into the coronary sinus;
releasing an anchor, which is fixed to the catheter, so as to anchor the catheter to the stent; and
administering the therapeutic substance from the catheter into the coronary sinus.

28. The method according to claim 27, wherein inserting the catheter comprises passing a distal end of the catheter through a constricted opening in the stent, and wherein releasing the anchor comprises inflating a balloon that is attached to the distal end of the catheter after the distal end has passed through the constricted opening.

29. The method according to claim 27, wherein releasing the anchor comprises advancing one or more wires from a distal end of the catheter so as to engage the stent, and then advancing the catheter distally along the wires to a position within the stent.

30. Apparatus for delivering a therapeutic substance in a coronary sinus of a patient, comprising:
a catheter, which is adapted to be inserted through a blood vessel of the patient into the coronary sinus, and which has a lumen for administration of the therapeutic substance therethrough into the coronary sinus; and
a resilient anchor, which is fixed to the catheter, so as to anchor the catheter in the coronary sinus without blocking blood flow through the coronary sinus.

31. The apparatus according to claim 30, wherein the catheter has a distal end and a channel, and wherein the resilient anchor comprises at least one resilient wire, which is arranged to be passed through the channel so as to protrude from the distal end of the catheter and engage a blood vessel wall.

32. The apparatus according to claim 31, wherein the catheter has at least first and second channels, and wherein the at least one resilient wire comprises at least first and second wires, which are arranged to be passed respectively through the first and second channels so as to engage the blood vessel wall at two or more locations on different sides of the catheter.

33. The apparatus according to claim 31, wherein the at least one resilient wire comprises a shape memory material, which is held straight within the channel and is pre-formed to bend into a predetermined shape upon protruding from the distal end so as to engage the blood vessel wall.

34. The apparatus according to claim 33, wherein the predetermined shape comprises at least one of a loop, a spiral and a zigzag shape.

35. The apparatus according to claim 33, wherein the predetermined shape comprises a bend, and wherein the at least one resilient wire is adapted to bend from the catheter into a side vessel that joins the coronary sinus and to engage the wall of the side vessel.

36. The apparatus according to claim 31, wherein the channel has multiple exit holes at different locations along the distal end of the catheter, and is arranged to permit an operator of the apparatus to choose one of the exit holes, and to advance the at least one resilient wire through the chosen exit hole so as to engage the blood vessel wall at a desired location.

37. The apparatus according to claim 31, wherein the channel has a proximal entrance, and wherein the at least one resilient wire has a proximal end and comprises a handle, which is fixed to the proximal end of the at least one resilient wire for use by an operator of the apparatus in manipulating the wire in the channel, so that the handle engages the proximal entrance of the channel when the at least one resilient wire is fully extended from the distal end of the catheter.

38. The apparatus according to claim 31, wherein the channel has a proximal end, and wherein the catheter comprises a locking mechanism for locking the at least one resilient wire in place at the proximal end of the channel after the at least one resilient wire has engaged the blood vessel wall so as to prevent relative axial movement between the catheter and the at least one resilient wire.

39. The apparatus according to claim 38, wherein the locking mechanism comprises a spring clip, which is arranged to press against the at least one resilient wire.

40. The apparatus according to claim 38, wherein the locking mechanism comprises a locking screw, which is arranged to engage the at least one resilient wire within the channel.

41. The apparatus according to claim 31, wherein the catheter comprises a distal end, which comprises multiple parallel leaves, and which are adapted to open outward to at least partially block the blood flow through the coronary sinus during the administration of the therapeutic substance.

42. The apparatus according to claim 41, and comprising a balloon at the distal end of the catheter, which is inflatable in order to push the leaves apart.

43. The apparatus according to claim 30, and comprising a flexible sheath, which is arranged to enclose the catheter while containing the resilient anchor, and to be advanced with the catheter through the blood vessel, wherein the flexible sheath is adapted to permit the catheter to be shifted distally relative to the sheath so as to release the anchor, causing the anchor to engage a blood vessel wall.

44. The apparatus according to claim 43, wherein the anchor comprises one or more resilient wires, which are preformed so as to spring outward into a predetermined shape when released from the sheath.

45. The apparatus according to claim 44, wherein the predetermined shape comprises at least one of a spiral, a loop and a hook shape.

46. The apparatus according to claim 43, wherein the catheter comprises a distal end, which comprises multiple parallel leaves formed from a resilient material, and which serve as the resilient anchor, such that upon shifting the catheter distally relative to the sheath, the leaves bend outward and engage the wall of the coronary sinus.

47. The apparatus according to claim 46, wherein the leaves are arranged, upon bending outward, to at least partially block blood flow through the coronary sinus.

48. The apparatus according to claim 47, wherein the leaves are constructed so as to deform in response to excessive blood pressure in the coronary sinus, thereby permitting blood to flow out of the coronary sinus.

49. The apparatus according to claim 30, wherein the resilient anchor is arranged to anchor the catheter by engaging a stent implanted in the coronary sinus.

50. Apparatus for delivering a therapeutic substance in a coronary sinus of a patient, comprising:
a catheter, which is adapted to be inserted through a blood vessel of the patient into the coronary sinus, and which has a lumen for administration of the therapeutic substance therethrough into the coronary sinus, and which comprises a distal end comprising multiple parallel leaves formed from a pliable material; and
a flexible sheath, which is arranged to enclose the catheter so as to contain the flexible leaves, and to be advanced with the catheter through a blood vessel into the coronary sinus, wherein the flexible sheath is adapted to permit the catheter to be shifted distally relative to the sheath so as to release the parallel leaves from the sheath and to cause the leaves to bend outward so that the leaves anchor the catheter in the coronary sinus.

51. The apparatus according to claim 50, wherein the leaves are adapted to bend outward under a pressure of blood flow in the coronary sinus.

52. The apparatus according to claim 50, and comprising a balloon at the distal end of the catheter, which is inflatable so as to push the leaves outward.

53. The apparatus according to claim 50, wherein the leaves are arranged, upon bending outward, to at least partially block the blood flow through the coronary sinus.

54. Apparatus for delivering a therapeutic substance in a coronary sinus of a patient, comprising:
a catheter, which is adapted to be inserted through a blood vessel of the patient into the coronary sinus, and which has a lumen for administration of the therapeutic substance therethrough into the coronary sinus; and
one or more balloons, which are fixed to the catheter, and which are inflatable so as to anchor the catheter in the coronary sinus without blocking blood flow through the coronary sinus.

55. The apparatus according to claim 54, and comprising at least one additional balloon, which is fixed to the catheter and is inflatable so as to adjustably block the blood flow through the coronary sinus while the catheter is anchored in the coronary sinus.

56. Apparatus for delivering a therapeutic substance in a coronary sinus of a patient, comprising:
a stent, which is adapted to be implanted within the coronary sinus;
a catheter, which is adapted to be inserted through a blood vessel of the patient into the coronary sinus, and which has a lumen for administration of the therapeutic substance therethrough into the coronary sinus; and
an anchor, which is fixed to the catheter, and is arranged to anchor the catheter to the stent.

57. The apparatus according to claim 56, wherein the stent contains a constricted opening, and wherein the catheter comprises a distal end that is adapted to pass through the constricted opening in the stent, and wherein the anchor comprises a balloon, which is attached to the distal end of the catheter and is arranged to be inflated after the distal end of the catheter has passed through the constricted opening.

58. The apparatus according to claim 56, wherein the catheter comprises a distal end, and wherein the anchor comprises one or more wires, which are arranged to be advanced from the distal end of the catheter so as to engage the stent, and wherein the catheter is arranged to be advanced distally along the wires to a position within the stent.
